(19) United States
(12) Patent Application Publication
(10) Pub. No.: US 2014/0136118 A1
(43) Pub. Date: May 15, 2014

(54) METHODS FOR EVALUATING CANINE PHYSICAL ACTIVITY

(71) Applicant: NESTEC SA, Vevey (CH)

(72) Inventors: Brian Michael Zanghi, Ballwin, MO (US); Robert Tochterman, St. Louis, MO (US); Shawn Brain, St. Louis, MO (US); Arleigh Reynolds, Salcha, AK (US); Steven S. Hannah, Chesterfield, MO (US); Ebenezer Satyaraj, Wildwood, MO (US)

(73) Assignee: NESTEC SA, Vevey (CH)

(21) Appl. No.: 14/076,881

(22) Filed: Nov. 11, 2013

Related U.S. Application Data
(60) Provisional application No. 61/726,156, filed on Nov. 14, 2012.

(51) Int. Cl. A61B 5/11

(52) U.S. Cl. CPC A61B 5/1118
USPC 702/19

(57) ABSTRACT

The invention provides methods and devices for evaluating and tracking canine physical activity by determining for a canine (1) the duration of an activity or exercise; the speed of the activity or exercise based on the type of activity or exercise pace, e.g., walking, trotting, cantering, or running; a breed category factor to accommodate speed differences in distance travelled because of differences in body size and stride length; and an age factor and (2) calculating physical activity points (PAP) for the canine using an equation designed to accept input for the variables in (1) and use those variables to calculate PAP. The results from the equation are used to evaluate the physical activity of the canine and, based upon the evaluation, alter, if required, the canine's physical activity in a way that benefits the canine, particularly the canine's health.

### CROSS REFERENCE TO RELATED APPLICATIONS

This application claims priority to U.S. Provisional Application No. 61/726,156 filed Nov. 14, 2012, the disclosure of which is incorporated herein by this reference.

### BACKGROUND OF THE INVENTION

1. Field of the Invention

This invention relates generally to evaluating animal physical activity and particularly to methods for evaluating and tracking canine physical activity.

2. Description of the Prior Art

The benefits of animal physical activity are well known. Regular physical activity is one of the most important things animals can do to maintain and improve good health. Physical activity helps control weight, reduce the risk of cardiovascular disease, reduce the risk for type 2 diabetes and metabolic syndrome, reduce the risk of some cancers, strengthen bones and muscles, improve mental health and mood, improve the ability to do daily activities, and increases longevity. However, evaluating and tracking animal physical activity and achieving the level of physical activity required to obtain these benefits is neither easy nor convenient. There is, therefore, a need for new methods for evaluating and tracking animal physical activity and using the information obtained by evaluating and tracking physical activity for the benefit of the animal.

### SUMMARY OF THE INVENTION

It is, therefore, an object of the present invention to provide methods for evaluating and tracking canine physical activity.

It is another object of the invention to provide devices useful for evaluating and tracking canine physical activity.

These and other objects are achieved using novel methods and devices for evaluating and tracking canine physical activity. Generally, an owner or caregiver for a canine evaluates and/or tracks the canine's physical activity by determining for a canine (1) the duration of an activity or exercise; the speed of the activity or exercise based on the type of activity or exercise pace, e.g., walking, trotting, cantering, or running; a breed category factor to accommodate speed differences in distance travelled because of differences in body size and stride length; and an age factor and (2) calculating physical activity points (PAP) for the canine using an equation designed to accept input for the variables in (1) and use those variables to calculate PAP. The results from the equation are used to evaluate the physical activity of the canine and, based upon the evaluation, alter, if required, the canine's physical activity in a way that benefits the canine, particularly the canine's health.

Other and further objects, features, and advantages of the present invention will be readily apparent to those skilled in the art.

### DETAILED DESCRIPTION OF THE INVENTION

### Definitions

The term “canine” means domesticated dogs or other canine animals that could benefit from evaluating or tracking of their physical activity and the information gathered by such evaluation and tracking.

The symbol “*” in the formulas herein means that the elements on each side of the “*” are multiplied, e.g., (1.2*minutes) means that the number of minutes is multiplied by 1.2.

The term “tablet PC” means a wireless, portable personal computer comprising a touch screen interface and having internet access.

The term “smartphone” means a cellular telephone comprising at least one software application and having internet access.

The term “smartphone application” means a software program that can be used on a smartphone, a tablet PC, other similar electronic device.

The term “walking” means that a canine is engaging in a walking activity or exercise by moving at a speed of 2.5 to 4.5 miles per hour (mph). Generally, all canines follow the same general pattern of movement when walking. Each leg alternately reaches forward, first on front leg, then the diagonal rear leg, then the other front leg and the other rear leg. This happens in rapid succession and the weight is generally borne on three of the limbs at any given time.

The term “trotting” means that a canine is engaging in a trotting activity or exercise by moving at a speed of from 4.6 mph to 7.7 mph. Generally, all canines follow the same general pattern of movement when trotting. Diagonal pairs of legs bear weight alternately so that there are two moments of weight bearing. Diagonal limbs move forward at the same time. That is, the dog's right front leg and the left rear leg move forward together followed by the left front leg and the right rear leg, which also move together.

The term “cantering” means that a canine is engaging in a cantering activity or exercise by moving a speed of from 7.8 mph to 12.5 mph. Generally, all canines follow the same general pattern of movement when cantering. The rear legs land almost at the same time, with one leg just ahead of the other. The leg that lands in front of the other is called the lead leg. The front lead leg bears the entire weight of the dog while the rear legs are swinging forward and thus is usually the dog's stronger leg.

The term “running” means that a canine is engaging in a running activity or exercise by moving a speed of greater than 12.6 mph. Generally, all canines follow the same general pattern of movement when running. There are four moments of weight-bearing; one rear leg lands followed by the other rear leg, then one front leg lands followed by the other.

This invention is not limited to the particular methodology and protocols described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise, e.g., reference to “a method” or “a smartphone” includes a plurality of such methods or smartphones. Similarly, the words “comprise”, “comprises”, and “comprising” are to be interpreted inclusively rather than exclusively.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents, patent applications, and publications mentioned herein are incorporated herein by reference to the extent allowed by law for the purpose of describing and disclosing the processes, techniques, procedures, technology, articles, and other devices and methods disclosed therein that might be used with the present invention. However, nothing herein is to be construed as an admission that the invention is not entitled to antedate such disclosure by virtue of prior invention.

### The Invention

In one aspect, the invention provides a method for evaluating or tracking canine physical activity. The method comprises (1) determining for the canine all but one variable selected from physical activity points (PAP), the duration of an activity (A), the speed of the activity based on the type of activity pace (S); (2) determining for the canine a breed category factor to accommodate speed differences in distance travelled because of differences in body size and stride length (B) and an age factor (C_{Breed Age}); and (3) calculating the undetermined variable for the canine using the equation PAP=(X+Z)*B (Equation 1), where:

X=(A*S)/60 and Z=(X*C_{Breed Age}), where:

A=activity duration minutes;

S=activity velocity, wherein S=3 if the activity is walking, S=5 if the activity is trotting, S=8 if the activity is cantering, and S=13 if the activity is running;

C_{Breed Age}=Age factor depending on breed, calculated as follows:

C_{Toy}=10(Y)−100 if Y>10.0 or C_{Toy}=0 if Y<10.0;

C_{Small}=10(Y)−90 if Y>9.0 or C_{Small}=0 if Y<9.0;

C_{Medium}=10(Y)−80 if Y>8.0 or C_{Medium}=0 if Y<8.0;

C_{Large}=10(Y)−70 if Y>7.0 or C_{Large}=0 if Y<7.0;

C_{Giant}=10(Y)−60 if Y>6.0 or C_{Giant}=0 if Y<6.0 where Y=years of age;

B=breed factor coefficient, wherein B=1.56 if the canine breed is toy, B=1.19 if the canine breed is small, B=1.0 if the canine breed in medium, B=0.81 if the canine breed is large, and B=0.46 if the canine breed is giant.

In Equation 1, the constants associated with C_{Breed Age} and B vary by plus or minus 20%, preferably plus or minus 10%, more preferably plus or minus 5%. For example, in the equation C_{Toy}=10(Y)−100 if Y>10.0, the constants 10 and 100 can vary from 8 to 12 and from 80 to 120, respectively.

In general, any undetermined variable calculated using Equation 1 is used for any suitable or desirable purpose, e.g., evaluating and/or tracking a canine's physical activity. For example, calculated physical activity points can be used to evaluate and track the physical activity of a particular canine for a particular activity at a particular time or over an extended period, e.g., from day to day, week to week, month to month, and the like. These calculations can be used to establish guidelines or goals for the canine and guide the canine's owner or caregiver when establishing a physical activity program for the canine, e.g., a physical activity program based on type and duration of activity designed to promote health and wellness for the canine. Also, physical activity points can be used to compare the physical activity points calculated for a particular canine to known physical activity points characteristic of the same or similar types of canines with a known health status, e.g., canines of the same age, size, breed, and the like that are in good health. Then, the physical activity points can be used to determine various activities and the level of such activities for the canine such that the canine can achieve the same or similar physical activity points characteristic of the same or similar canines.

In one embodiment, a canine's physical activity points are calculated and compared to a known set of desirable canine physical activity points that have been determined for a group of the same or similar canines having desirable physical and health characteristics, e.g., a desirable weight and free from maladies. For example, the physical activity points for known canine breeds, sizes, ages, and the like can be calculated and provided to an owner or caregiver, e.g., in a table or an online database. Then, the desirable physical activity points for a canine under the care of the owner or caregiver can be determined by comparing the canine's characteristics to those in the table. Following such determination, Equation 1 can be used to determine an activity duration and an activity velocity based on the desired physical activity points. For example, if the desired physical activity points for a certain canine is a number J, the type of exercise, e.g., running, and the time the canine must run to achieve J can be calculated and the canine can be put on an exercise regime to achieve the desired physical activity points. Depending on needs or desires, the type of physical activity and the time for engaging in such physical activity can be varied, so long as the desired physical activity points are achieved.

In another embodiment, a canine's physical activity points are calculated for a known physical activity for a known duration and compared to the desirable physical activity points for the canine, e.g., from the table or database. Then, the physical activity of the canine can be evaluated to see if the canine is engaging in sufficient physical activity to promote good health. If not, the canine's physical activity can be adjusted to achieve the desirable physical activity points.

In a further embodiment, a canine's physical activity points are determined when the canine engages in little or no exercise. Then, the canine's physical activity points are determined when the canine engages in moderate, vigorous, or other level of physical activity for a certain duration, particularly when the canine achieves a better health status or other desirable status following the change in physical activity. Based upon a comparison of the calculated physical activity points, a canine owner or caregiver can calculate the amount and type of various physical activities that are required to achieve desirable physical activity points. For example, if a canine with known and desirable physical activity points is in a situation where the canine must exercise by walking, the duration of the walking activity can be determined. Many other such calculations can be performed depending on the situation for the canine.

For a canine based on size, i.e., toy, small, medium, large, or giant, this is a characterization known to or easily determined by skilled artisans. However, what is not easily known is how breed influences the speed of travel for a particular pace of locomotion. The skilled artisan will appreciate that stride length or body length will vary for each breed and within breed, and will influence the speed of travel at a given pace. For this example, body length was used to adjust for differences in speed for different breeds at a given pace.

In another aspect, the present invention provides a device useful for evaluating or tracking canine physical activity. The device comprises a means for evaluating or tracking canine physical activity that uses a method for evaluating or tracking canine physical activity comprising (1) determining for the canine all but one variable selected from physical activity points (PAP), the duration of an activity (A), the speed of the activity based on the type of activity pace (S); (2) determining for the canine a breed category factor to accommodate speed differences in distance travelled because of differences in body size and stride length (B) and an age factor (C_{Breed Age}); and (3) calculating the undetermined variable for the canine using the equation PAP=(X+Z)*B (Equation 1), where:

X=(A*S)/60 and Z=(X*C_{Breed Age}), where:

A=activity duration minutes;

S=activity velocity, wherein S=3 if the activity is walking, S=5 if the activity is trotting, S=8 if the activity is cantering, and S=13 if the activity is running;

C_{Breed Age}=Age factor depending on breed, calculated as follows:

C_{Toy}=10(Y)−100 if Y>10.0 or C_{Toy}=0 if Y<10.0;

C_{Small}=10(Y)−90 if Y>9.0 or C_{Small}=0 if Y<9.0;

C_{Medium}=10(Y)−80 if Y>8.0 or C_{Medium}=0 if Y<8.0;

C_{Large}=10(Y)−70 if Y>7.0 or C_{Large}=0 if Y<7.0;

C_{Giant}=10(Y)−60 if Y>6.0 or C_{Giant}=0 if Y<6.0 where Y=years of age;

B=breed factor coefficient, wherein B=1.56 if the canine breed is toy, B=1.19 if the canine breed is small, B=1.0 if the canine breed in medium, B=0.81 if the canine breed is large, and B=0.46 if the canine breed is giant, wherein the constants in Equation 1 for C_{Breed Age} and B vary by plus or minus 20%, or 10% or 5%.

The means for evaluating and/or tracking canine physical activity is any suitable means for accepting input relating to the variables in Equation 1, performing calculations using the input and Equation 1, and displaying the calculated results in a form useable by a canine owner or caregiver, e.g., a printout, a monitor display, or a smartphone display. In various embodiments, the means is a software program such as a smartphone application designed to calculate results based on Equation 1 variables, e.g., calculating physical activity points using Equation 1 and the other Equation 1 variables or calculating one of the other Equation 1 variables based on physical activity points and the remaining variables.

In various embodiments, the device is an electronic device programmed with a software program designed to permit a user to input data about the variables and calculate the physical activity points using Equation 1. In preferred embodiments, the device is a computer, programmable calculator, tablet PC, smartphone, or similar device. In a preferred embodiment, the device is a smartphone or tablet PC, e.g., Apple® brand of IPhone, IPod, or IPad or their equivalents made by Amazon®, Samsung®, Google® or other manufacturers. The device can use any suitable operating system compatible with the contemplated software program, e.g., Microsoft Windows®, Mac OS X®, or Android® operating systems. In another embodiment, the device is an electronic device programmed with software program designed to permit a user to input data about physical activity points and the variables in Equation, except for one variable, and calculate the remaining variable using Equation 1.

In various embodiments, the device may be programmed with the software or may be in communication with other devices that are programmed with the software. For example, a first device may be a computer that has a wired or wireless connection to the internet. The computer receives input and communicates the input via the internet to a second device that is programmed to perform the calculations using Equation 1, e.g., a second computer. The second device performs the calculation and communicates via the internet the calculated result to the first device, which displays the result to the owner or caregiver that provided the input. The first device could be a smartphone, a tablet PC, or any similar device that can receive the input and communicate with the second device.

In preferred embodiments, the device accepts input from a user in the form of Equation 1 variables other than PAP and uses Equation 1 to calculate physical activity points.

In another aspect, the present invention provides a smartphone application suitable for use on a smartphone, tablet PC, or similar device. The smartphone application comprises (i) programmable code that calculates a result using a method for evaluating or tracking canine physical activity comprising (1) determining for the canine all but one variable selected from physical activity points (PAP), the duration of an activity (A), the speed of the activity based on the type of activity pace (S); (2) determining for the canine a breed category factor to accommodate speed differences in distance travelled because of differences in body size and stride length (B) and an age factor (C_{Breed Age}); and (3) calculating the undetermined variable for the canine using the equation PAP=(X+Z)*B (Equation 1), where:

X=(A*S)/60 and Z=(X*C_{Breed Age}), where:

A=activity duration minutes;

S=activity velocity, wherein S=3 if the activity is walking, S=5 if the activity is trotting, S=8 if the activity is cantering, and S=13 if the activity is running;

C_{Breed Age}=Age factor depending on breed, calculated as follows:

C_{Toy}=10(Y)−100 if Y>10.0 or C_{Toy}=0 if Y<10.0;

C_{Small}=10(Y)−90 if Y>9.0 or C_{Small}=0 if Y<9.0;

C_{Medium}=10(Y)−80 if Y>8.0 or C_{Medium}=0 if Y<8.0;

C_{Large}=10(Y)−70 if Y>7.0 or C_{Large}=0 if Y<7.0;

C_{Giant}=10(Y)−60 if Y>6.0 or C_{Giant}=0 if Y<6.0 where Y=years of age;

B=breed factor coefficient, wherein B=1.56 if the canine breed is toy, B=1.19 if the canine breed is small, B=1.0 if the canine breed in medium, B=0.81 if the canine breed is large, and B=0.46 if the canine breed is giant, wherein the constants in Equation 1 for C_{Breed Age} and B vary by plus or minus 20%, or 10% or 5%;

(ii) an interface that accepts user input in the form of variables in Equation 1, and

(iii) a means for displaying the calculated result on a smartphone, tablet PC, or similar device.

In a further aspect, the invention provides an electronic computing device comprising the smartphone application of the invention. In preferred embodiments, the electronic computing device is a smartphone or tablet PC, preferably a smartphone.

In another aspect, the present invention provides a means for communicating information about or instructions for using the methods and devices of the present invention to track canine physical activity. The communicating means comprises a document, digital storage media, optical storage media, audio presentation, or visual display containing the information or instructions. Preferably, the communication is a displayed website or a brochure, product label, package insert, advertisement, or visual display containing such information or instructions. Useful information and instructions include, but are not limited to, contact information for canines or their caregivers to use if they have a question about the invention and its use and how to use the present invention to calculate physical activity points. The communication means is useful for instructing a canine owner or caregiver on the benefits of using the present invention.

In the specification, there have been disclosed typical preferred embodiments of the invention and, although specific terms are employed, they are used in a generic and descriptive sense only and not for purposes of limitation, the scope of the invention being set forth in the following claims. Obviously many modifications and variations of the present invention are possible in light of the above teachings. It is therefore to be understood that within the scope of the appended claims the invention may be practiced otherwise than as specifically described.

What is claimed is:

1. A method for evaluating or tracking canine physical activity comprising:
(1) determining for the canine all but one variable selected from physical activity points (PAP), the duration of an activity (A), the speed of the activity based on the type of activity pace (S);
(2) determining for the canine a breed category factor (B) and an age factor (C_{Breed Age}); and
(3) calculating the undetermined variable for the canine using the equation PAP=(X+Z)*B (Equation 1), where:
X=(A*S)/60 and Z=(X*C_{Breed Age}), where:
A=activity duration minutes;
S=activity velocity, wherein S=3 if the activity is walking, S=5 if the activity is trotting, S=8 if the activity is cantering, and S=13 if the activity is running;
C_{Breed Age}=Age factor depending on breed, calculated as follows:
C_{Toy}=10(Y)−100 if Y>10.0 or C_{Toy}=0 if Y<10.0;
C_{Small}=10(Y)−90 if Y>9.0 or C_{Small}=0 if Y<9.0;
C_{Medium}=10(Y)−80 if Y>8.0 or C_{Medium}=0 if Y<8.0;
C_{Large}=10(Y)−70 if Y>7.0 or C_{Large}=0 if Y<7.0;
C_{Giant}=10(Y)−60 if Y>6.0 or C_{Giant}=0 if Y<6.0 where Y=years of age;
B=breed factor coefficient, wherein B=1.56 if the canine breed is toy, B=1.19 if the canine breed is small, B=1.0 if the canine breed in medium, B=0.81 if the canine breed is large, and B=0.46 if the canine breed is giant, wherein the constants in Equation 1 for C_{Breed Age} and B vary by plus or minus 20%.

2. A device useful for evaluating or tracking canine physical activity comprising a means for evaluating or tracking canine physical activity that uses a method comprising:
(1) determining for the canine all but one variable selected from physical activity points (PAP), the duration of an activity (A), the speed of the activity based on the type of activity pace (S);
(2) determining for the canine a breed category factor (B) and an age factor (C_{Breed Age}); and
(3) calculating the undetermined variable for the canine using the equation PAP=(X+Z)*B (Equation 1), where:
X=(A*S)/60 and Z=(X*C_{Breed Age}), where:
A=activity duration minutes;
S=activity velocity, wherein S=3 if the activity is walking, S=5 if the activity is trotting, S=8 if the activity is cantering, and S=13 if the activity is running;
C_{Breed Age}=Age factor depending on breed, calculated as follows:
C_{Toy}=10(Y)−100 if Y>10.0 or C_{Toy}=0 if Y<10.0;
C_{Small}=10(Y)−90 if Y>9.0 or C_{Small}=0 if Y<9.0;
C_{Medium}=10(Y)−80 if Y>8.0 or C_{Medium}=0 if Y<8.0;
C_{Large}=10(Y)−70 if Y>7.0 or C_{Large}=0 if Y<7.0;
C_{Giant}=10(Y)−60 if Y>6.0 or C_{Giant}=0 if Y<6.0 where Y=years of age;
B=breed factor coefficient, wherein B=1.56 if the canine breed is toy, B=1.19 if the canine breed is small, B=1.0 if the canine breed in medium, B=0.81 if the canine breed is large, and B=0.46 if the canine breed is giant, wherein the constants in Equation 1 for C_{Breed Age} and B vary by plus or minus 20%.

3. The device of claim 2 wherein the means is a software program.

4. The device of claim 3 wherein the software program is a smartphone application.

5. The device of claim 2 wherein the device is selected from a computer, a programmable calculator, a smartphone, or a tablet PC.

6. The device of claim 2 wherein the device is a tablet PC or a smartphone.

7. The device of claim 2 wherein the device is a smartphone.

8. A smartphone application comprising:
(i) programmable code that calculates a result using a method for evaluating or tracking canine physical activity comprising (1) determining for the canine all but one variable selected from physical activity points (PAP), the duration of an activity (A), the speed of the activity based on the type of activity pace (S); (2) determining for the canine a breed category factor (B) to accommodate speed differences in distance travelled because of differences in body size and stride length and an age factor (C_{Breed Age}); and (3) calculating the undetermined variable for the canine using the equation PAP=(X+Z)*B (Equation 1), where:
X=(A*S)/60 and Z=(X*C_{Breed Age}), where:
A=activity duration minutes;
S=activity velocity, wherein S=3 if the activity is walking, S=5 if the activity is trotting, S=8 if the activity is cantering, and S=13 if the activity is running;
C_{Breed Age}=Age factor depending on breed, calculated as follows:
C_{Toy}=10(Y)−100 if Y>10.0 or C_{Toy}=0 if Y<10.0;
C_{Small}=10(Y)−90 if Y>9.0 or C_{Small}=0 if Y<9.0;
C_{Medium}=10(Y)−80 if Y>8.0 or C_{Medium}=0 if Y<8.0;
C_{Large}=10(Y)−70 if Y>7.0 or C_{Large}=0 if Y<7.0;
C_{Giant}=10(Y)−60 if Y>6.0 or C_{Giant}=0 if Y<6.0 where Y=years of age;
B=breed factor coefficient, wherein B=1.56 if the canine breed is toy, B=1.19 if the canine breed is small, B=1.0 if the canine breed in medium, B=0.81 if the canine breed is large, and B=0.46 if the canine breed is giant, wherein the constants in Equation 1 for C_{Breed Age} and B vary by plus or minus 20%.
(ii) an interface that accepts user input in the form of variables in Equation 1, and
(iii) a means for displaying the calculated result on a smartphone, tablet PC, or similar device.

9. An electronic device comprising the smartphone application of claim 8.

10. The electronic device of claim 9 wherein the electronic device is selected from a smartphone or a tablet PC.

11. The electronic device of claim 9 wherein the electronic device is a smartphone.

12. A means for communicating information about or instructions for using the methods and devices of the present invention to track canine physical activity comprising a document, digital storage media, optical storage media, audio presentation, or visual display containing the information or instructions.

13. The means for communicating of claim 12 selected from the group consisting of a displayed website, brochure, product label, package insert, advertisement, and visual display.
